(19) United States
(12) Patent Application Publication
(10) Pub. No.: US 2014/0179645 A1
(43) Pub. Date: Jun. 26, 2014

(54) ANTIMICROBIAL COMPOSITIONS

(71) Applicant: THE PISCES GROUP LLC, Laguna Niguel, CA (US)

(72) Inventor: Douglas Charles Arndt, Ventura, CA (US)

(73) Assignee: THE PISCES GROUP LLC, Laguna Niguel, CA (US)

(21) Appl. No.: 14/101,494

(22) Filed: Dec. 10, 2013

Related U.S. Application Data
(60) Provisional application No. 61/740,501, filed on Dec. 21, 2012.

(51) Int. Cl. A01N 55/02; A61L 2/18; A01N 37/10; A01N 37/40; A01N 31/16; A01N 43/42

(52) U.S. Cl. CPC A01N 55/02; A01N 31/16; A01N 43/42; A01N 37/10; A01N 37/40; A61L 2/18
USPC 514/163; 514/500; 422/28

(57) ABSTRACT

Described herein are antimicrobial compositions, methods of making thereof, a kit comprising said compositions, and methods for removing microorganisms from surfaces by using said compositions. In one aspect, the antimicrobial composition is a kit composed of a first composition that comprises a dissolved metal salt and a second composition that comprises a ligand, wherein the ligand comprises (i) a residue of antibacterial agent and (ii) a residue of an antifungal agent, wherein the antibacterial agent and/or antifungal agent comprises at least one aryl group. These compositions can further comprise (a) surfactants and/or emulsifiers, (b) thickeners, and (c) solvents or carriers such as water, propylene glycol, and glycerin. In some aspects, these compositions can also comprise fragrances, humectants, emollients, essential oils, plant extracts, and other natural products. Optional additives that are known in the art may also be included.

### CROSS REFERENCE TO RELATED APPLICATIONS

This application claims priority upon U.S. provisional application Ser. No. 61/740,501, filed Dec. 21, 2012. This application is hereby incorporated by reference in its entirety for all of its teachings.

### BACKGROUND

Bacterial, fungal, and viral infections have long been a subject of concern for public health officials as well as for members of the general population. Cases of food poisoning and nosocomial infections are on the rise. Further, many hours of work are lost each year when employees contract communicable diseases, which has a significant economic impact on businesses.

One way to reduce or prevent the transmission of many diseases is to sanitize the hands, thereby killing and/or removing the infectious microorganisms that cause said diseases. Another way to reduce or prevent the transmission of disease is to disinfect inanimate surfaces and/or substrates that come into contact with individuals who may be carrying disease-causing microorganisms. Two strategies are commonly employed for hand sanitization: alcohol-based hand sanitizers and washing the hands with soap and water. The Centers for Disease Control and Prevention recommend washing with soap and clean, running water as the best method for sanitizing the hands. Alcohol-based hand sanitizers may be used, but are unable to remove visible soil which may harbor additional microorganisms and/or resistant spores. Further, many commercial alcohol-based hand sanitizers may contain less than the minimum 60 percent concentration of alcohol recommended by the CDC.

Hand soaps may be rendered more effective at controlling the spread of pathogenic microorganisms if they are formulated with an additional antimicrobial ingredient. A variety of such ingredients are known in the art; however, this approach has drawbacks as well. Bisbiguanide additives such as chlorhexidine may be deactivated by anionic surfactants. Widespread use of halogenated phenolic additives such as triclosan has led to the emergence of resistant strains of bacteria. Additionally, triclosan breakdown products may be environmentally harmful. Many household and industrial cleaning compositions also contain additives such as chlorhexidine or triclosan, and suffer from the same drawbacks.

It would therefore be desirable to develop antimicrobial compositions such as, for example, hand soaps or cleaning compositions, that are mild, that have a higher level of activity than existing compositions, that are active against a broad spectrum of microbial pathogens, that cannot be deactivated by the presence of other components such as surfactants, and that may be stored for long periods of time without a loss in efficacy due to precipitation of active components. The present invention accomplishes these goals, as will be demonstrated below in the detailed description and examples.

### SUMMARY

Described herein are antimicrobial compositions, methods of making thereof, a kit comprising said compositions, and methods for removing microorganisms from surfaces by using said compositions. In one aspect, the antimicrobial composition is a kit composed of a first composition that comprises a dissolved metal salt and a second composition that comprises a ligand, wherein the ligand comprises (i) a residue of antibacterial agent and (ii) a residue of an antifungal agent, wherein the antibacterial agent and/or antifungal agent comprises at least one aryl group. These compositions can further comprise (a) surfactants and/or emulsifiers, (b) thickeners, and (c) solvents or carriers such as water, propylene glycol, and glycerin. In some aspects, these compositions can also comprise fragrances, humectants, emollients, essential oils, plant extracts, and other natural products. Optional additives that are known in the art may also be included.

Additional advantages of the compositions, methods, and articles described herein will be set forth in part in the description that follows, and in part will be apparent from the description. The advantages of the compositions, methods, and articles described herein will be realized and attained by means of the elements and combinations particularly pointed out in the appended claims. It is to be understood that both the foregoing general description and the following detailed description are exemplary and explanatory only and are not restrictive of the compositions, methods, and articles described herein, as claimed.

### DETAILED DESCRIPTION

The compositions, methods, and articles described herein can be understood more readily by reference to the following detailed description. It is also to be understood that the terminology used herein is for the purpose of describing particular aspects only and is not intended to be limiting.

It must be noted that, as used in the specification and the appended claims, the singular forms “a,” “an,” and “the” include plural referents unless the context clearly dictates otherwise. Thus, for example, reference to “a humectant” includes mixtures of two or more humectants.

“Optional” or “optionally” means that the subsequently described event or circumstance can or cannot occur, and that the description includes instances where the event or circumstance occurs and instances where it does not. For example, the phrase “optionally includes an emollient” means that an emollient can or cannot be included.

As used herein, the term “about” is used to provide flexibility to a numerical range endpoint by providing that a given value may be “a little above” or “a little below” the endpoint without affecting the desired result.

“Antimicrobial” refers to a substance that kills or inhibits the growth of microorganisms. In one aspect, the microorganism is a bacterium, a fungus, an archaeon, or a protozoan. In another aspect, the antimicrobial soap can additionally possess antiviral activity.

A “surfactant” is an organic compound that may be derived from a natural product, or may result from chemical modification of a natural product, or may be completely chemically synthesized. Surfactants typically contain hydrophilic head groups and hydrophobic tails. In one aspect, the head group is anionic, cationic, non-ionic, or zwitterionic. In another aspect, the tail is composed of a hydrocarbon or a glucoside. Surfactants alter the surface tension of liquids and may form micelles or bilayers in aqueous solution. Many applications of surfactants are known in the art; surfactants are, for example, commonly employed as emulsifiers, detergents, wetting agents, and other related uses.

“Humectants” are substances that promote water retention. “Emollients” are compounds which soften the skin by increasing the skin's hydration. Either humectants or emollients, or both, may be used as ingredients in cosmetic and/or cleansing compositions to prevent drying of the skin due to use of said compositions.

As used herein, a “thickener” is a compound which increases the viscosity of a solution. In one aspect, the thickener may be a polysaccharide or a derivative thereof. Thickeners may also help to stabilize emulsions.

As used herein, “nascent state” refers to a molecular-sized chelation complex with antimicrobial properties. The nascent state forms immediately upon the mixing of the two compositions described herein, the first of which contains a dissolved metal salt and the second of which contains a ligand capable of chelating the metal ion contained in the first composition.

As used herein, “colloidal chelate” refers to a colloidal-type complex formed from the nascent state as the nascent state complex aggregates over time. The colloidal chelate precipitates out of the dispersion medium and becomes visible and easy to wash off the substrate.

A “residue” of a chemical species, as used in the specification and appended claims, refers to the moiety that is the resulting product of the chemical species in a particular reaction scheme or subsequent formulation or chemical product, regardless of whether the moiety is actually obtained from the chemical species. For example, an antibacterial agent that contains at least one —OH group can be represented by the formula Y—OH, where Y is the remainder (i.e., residue) of the antibacterial agent.

The term “aryl group” as used herein is any group containing an aromatic group including, but not limited to, benzene, naphthalene, etc. The term “aryl” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

“Admixing” or “admixture” refers to a combination of two or components together wherein there is no chemical reaction or physical interaction. The terms “admixing” and “admixture” can also include the chemical reaction or physical interaction between any of the components described herein upon mixing to produce the composition. The components can be admixed alone, in water, in another solvent, or in a combination of solvents.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an illustration, a numerical range of “about 1 to about 5” should be interpreted to include not only the explicitly recited values of about 1 to about 5, but also to include individual values and sub-ranges within the indicated range. Thus, included in this numerical range are individual values such as 2, 3, and 4, and sub ranges such as from 1-3, from 2-4, and from 3-5, etc., as well as 1, 2, 3, 4, and 5, individually. The same principle applies to ranges reciting only one numerical value as a minimum or a maximum. Furthermore, such an interpretation should apply regardless of the breadth of the range or the characteristics being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. For example, if an antifungal agent is disclosed and discussed and a number of different antibacterial agents are discussed, each and every combination and permutation of antifungal agent and antibacterial agent that is possible is specifically contemplated unless specifically indicated to the contrary. For example, if a class of molecules A, B, and C are disclosed, as well as a class of molecules D, E, and F, and an example of a combination A+D is disclosed, then even if each is not individually recited, each is individually and collectively contemplated. Thus, in this example, each of the combinations A+E, A+F, B+D, B+E, B+F, C+D, C+E, and C+F, are specifically contemplated and should be considered disclosed from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered disclosed from disclosure of A, B, and C; D, E, and F; and the example combination of A+D. This concept applies to all aspects of this disclosure including, but not limited to, steps in methods of making and using the disclosed compositions. Thus, if there are a variety of additional steps that can be performed with any specific embodiment or combination of embodiments of the disclosed methods, and that each such combination is specifically contemplated and should be considered disclosed.

Throughout this application, where publications are referenced, the disclosures of these publications in their entireties are hereby incorporated by reference into this application in order to more fully describe the state of the art to which this invention pertains.

Described herein are antimicrobial compositions, methods of making thereof, a kit comprising said compositions, and methods for removing microorganisms from surfaces by using said compositions. In one aspect, the antimicrobial composition is a kit composed of a first composition that comprises a dissolved metal salt and a second composition that comprises a ligand, wherein the ligand comprises (i) a residue of antibacterial agent and (ii) a residue of an antifungal agent, wherein the antibacterial agent and/or antifungal agent comprises at least one aryl group. These compositions can further comprise (a) surfactants and/or emulsifiers, (b) thickeners, and (c) solvents or carriers such as water, propylene glycol, and glycerin. In some aspects, these compositions can also comprise fragrances, humectants, emollients, essential oils, plant extracts, and other natural products. Optional additives that are known in the art may also be included. Each component is described in detail below, as are methods of making and using the antimicrobial soap compositions.

In one aspect, a metal ion present in the first composition described herein may be chelated by a ligand. In a further aspect, the metal ion can be a transition metal. In another aspect, the transition metal can be copper, manganese, or zinc. In a particularly preferred aspect, the transition metal is copper.

In another aspect, the source of the metal ion in the first composition described herein is a metal salt. In a further aspect, the metal salt is dissolved in a solvent and dissociated into a metal cation and an anionic counter ion. The selection of the solvent can vary depending upon the metal complex that is used. In one aspect, the solvent in the first composition is water. The selection of the counterion can also vary. In the case when the kit is used in human applications, the counterion should not possess any deleterious properties to skin.

The counterion of the metal ion is generally selected so that the corresponding conjugate acid is not corrosive to the substrate of interest (e.g., skin). In certain aspects, the conjugate acid of the counterion can provide additional benefits. For example, if a gluconate salt of a transition metal is used, the gluconate anion can provide additional benefits for aged or damaged skin when it reacts with a hydrogen liberated from the antibacterial agent, from the antifungal agent, or from both, to form gluconic acid. In other aspects, the counterion can be edentates, a sugar (e.g., fructose), or an amino acid conjugate.

In one aspect, the metal salt is a copper salt. In a further aspect, the copper salt can be copper aspartate, copper carbonate, copper chlorophyllin, copper citrate, copper gluconate, copper oxalate, copper picolinate, and copper tartrate. In a preferred aspect, the copper salt is copper gluconate.

In an alternative aspect, the metal salt is a zinc salt. In a further aspect, the zinc salt can be zinc ascorbate, zinc aspartate, zinc butyrate, zinc carbonate, zinc carbonate hydroxide, zinc citrate, zinc formate, zinc gluconate, zinc glutamate, zinc glycerate, zinc glycinate, zinc lactate, zinc oxide, zinc picolinate, zinc propionate, zinc sulfide, and zinc tartrate.

In another aspect, the metal salt is a manganese salt. In a further aspect, the manganese salt can bemanganese acetate, manganese ascorbate, manganese carbonate, manganese citrate, manganese gluconate, and manganese lactate.

In one aspect, the metal salt is from about 0.1 to about 10 parts by weight of the first composition. In other aspects, the metal salt is about 0.1, about 1, about 2, about 3, about 4, about 5, about 6, about 7, about 8, about 9, or about 10 parts by weight of the first composition. In an alternative aspect, the metal salt is about 2 to about 8 parts by weight of the first composition. In a further aspect, the metal salt is about 4 to about 5 parts by weight of the first composition.

In one aspect, a ligand from the second composition forms a chelation complex with the metal ion from the first composition upon mixing of the two compositions. In one aspect, the ligand is the reaction product between the antibacterial agent and the antifungal agent. Each component is described in detail below.

In one aspect, the antibacterial agent comprises a benzoic acid compound. In a further aspect, the benzoic acid analog has the structure (I):

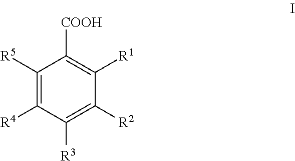

In one aspect, R¹, R², R⁴, and R⁵ are independently H, OH, F, I, Br, Cl, SH, NH₂, CN, alkyl, alkoxyl, NR, OR, NO₂, COR, CONR₂, CO₂, SO₃R and wherein R is independently H, alkyl, and alkoxyl groups. R³ is independently H, OH, F, I, Br, Cl, SH, CN, alkyl, alkoxyl, OR, NO₂, COR, CONR₂, CO₂R, SO₃R and wherein R is independently H, alkyl, and alkoxyl groups. In one aspect, alkyl groups can besaturated or unsaturated, linear or branched chain, substituted or unsubstituted alkyl groups. In a further aspect, substituted alkyls can beCH₂CO₂R, CH₂OR, CH₂OR, CH₂COR, and CH₂NR₂, where R is defined as above. In another aspect, alkoxyl groups can besaturated or unsaturated, linear or branched chain, substituted or unsubstituted alkoxyl groups

In one aspect, benzoic acid compounds are those wherein R¹, R², R⁴, and R⁵ are independently H, hydroxy, amino, diethylamino, dimethylamino, methyl, ethyl, propyl, butyl, ethoxy, methoxy, propoxy, butoxy, C(O)CH₃C(O)C₃H₇, C(O)C₄H₈, CO₂CH₃, CO₂C₃H₇, CH₂OH, CH₂OCH₃, CH₂OC₃H₇, COOH, chloro, fluoro, bromo, trifluoromethyl, nitro, and cyano and R³ is selected from the group consisting H, hydroxy, diethylamino, dimethylamino, methyl, ethyl, propyl, butyl, ethoxy, methoxy, propoxy, butoxy, C(O)CH₃C(O)C₃H₇, C(O)C₄H₈, CO₂CH₃, CO₂C₃H₇, CH₂OH, CH₂OCH₃, CH₂OC₃H₇, COOH, chloro, fluoro, bromo, trifluoromethyl, nitro, and cyano.

In another aspect, benzoic acid compounds are those wherein R¹-R⁵ are independently H, hydroxy, dimethylamino, methyl, ethyl, ethoxy, methoxy, C(O)CH₃C(O)C₃H₇, CO₂CH₃, CH₂OCH₃, CH₂OC₃H₇, COOH, chloro, iodo, bromo, trifluoromethyl, nitro, and cyano. In another aspect, benzoic acid compounds are those wherein R¹-R⁵ are independently H, hydroxy, methyl, methoxy, C(O)CH₃, CH₂OCH₃, COOH, chloro, iodo, nitro, thio, bromo, and cyano.

In one aspect, the benzoic acid compound is benzoic acid, salicylic acid, 2-nitrobenzoic acid, thiosalicylic acid, 2,6-dihydroxybenzoic acid, 3-hydroxybenzoic acid, 5-nitrosalicylic acid, 5-bromosalicylic acid, 5-iodosalicylic acid, 5-fluorosalicylic acid, 3-chlorosalicylic acid, 4-chlorosalicylic acid, 5-chlorosalicylic acid, phthalic acid, and combinations thereof.

In one aspect, the antifungal agent is a quinoline compound of formula (II)

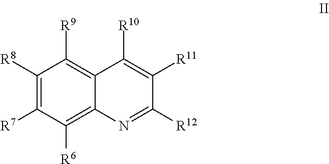

wherein R⁶-R¹² are independently H, OH, F, I, Br, Cl, SH, NH₂, CN, alkyl, alkoxyl, NR, OR, NO₂, COR, CONR₂, CO₂, SO₃R and wherein R is independently H, alkyl, and alkoxyl groups. In one aspect, R⁶ is a monoprotic substituent. In another aspect, R⁶ is OH and R⁷-R¹² are H (i.e., the antifungal agent is 8-hydroxyquinoline, also known as oxine).

In a further aspect, the ligand can be an 8-hydroxyquinoline derivative and a salicylate derivative or an 8-hydroxyquinoline derivative and a naphthoate derivative. In a still further aspect, the organic ligand may be selected from the group comprising 8-hydroxyquinoline salicylate, 8-hydroxyquinoline-5′-chlorosalicylate, 8-hydroxyquinoline-5′-iodosalicylate, 8-hydroxyquinoline-3′,5′-diiodosalicylate, 5-chloro-8-hydroxyquinoline-5′-chlorosalicylate, 5-chloro-8-hydroxyquinoline-3′,5′-dichlorosalicylate, 5-chloro-8-hydroxyquinoline-3′,5′-diiodosalicylate, 5-iodo-8-hydroxyquinoline-3′,5′-dichlorosalicylate, 5-iodo-8-hydroxyquinoline-3′,5′-dioodosalicylate, 8-hydroxyquinoline-3′-hydroxy-2′-naphthoate, 8-hydroxyquinoline-4′-chloro-3′-hydroxy-2′-naphthoate, 8-hydroxyquinoline-4′-bromo-3′-hydroxy-2′-naphthoate, 8-hydroxyquinoline-7′-bromo-3′-hydroxy-2′-naphthoate, 8-hydroxyquinoline-4′,7′-dibromo-3′-hydroxy-2′-naphthoate, 8-hydroxyquinoline-1′-hydroxy-2′-naphthoate, 8-hydroxyquinoline-4′-chloro-1′-hydroxy-2′-naphthoate, 8-hydroxyquinoline-2′-hydroxy-1′-naphthoate, 5-chloro-8-hydroxyquinoline-4′-bromo-3′-hydroxy-2′-naphthoate, 5-chloro-8-hydroxyquinoline-7′-bromo-3′-hydroxy-2′-naphthoate, 5-chloro-8-hydroxyquinoline-4′,7′-dibromo-3′-hydroxy-2′-naphthoate, 5-chloro-8-hydroxyquinoline-2′-hydroxy-1′-naphthoate, 5-iodo-8-hydroxyquinoline-4′-chloro-3′-hydroxy-2′-naphthoate, 5-iodo-8-hydroxyquinoline-4′-bromo-3′-hydroxy-2′-naphthoate, 5-iodo-8-hydroxyquinoline-7′-bromo-3′-hydroxy-2′-naphthoate, and 5-iodo-8-hydroxyquinoline-4′-chloro-1′-hydroxy-2′-naphthoate. In one aspect, the ligand is 8-hydroxyquinoline salicylate (CAS #2439-07-8).

In one aspect, the ligand is added to the second composition as a pre-formed salt comprising the antibacterial agent and the antifungal agent. In another aspect, the ligand is produced in situ by reacting the antibacterial agent with the antifungal agent during the preparation of the second composition.

In another aspect, the second composition comprises the ligand and one or more organic solvents. In another aspect, the organic solvent may also serve as a humectant or other substance that promotes water retention. In yet another aspect, the organic solvent may be glycerin, propylene glycol, or a mixture thereof.

In one aspect, the ligand is from about 1 to about 10 parts by weight of the second composition. In other aspects, the ligand is about 1, about 2, about 3, about 4, about 5, about 6, about 7, about 8, about 9, or about 10 parts by weight of the second composition. In an alternative aspect, the ligand is about 1 to about 5 parts by weight of the second composition. In a further aspect, the ligand is about 2 to about 5 parts by weight of the second composition.

In one aspect, either or both compositions disclosed herein may comprise one or more surfactants. In a further aspect, the surfactants may also act as emulsifiers and/or foaming agents. In an additional aspect, the surfactant can be a cationic, anionic, zwitterionic, or nonionic surfactant. In one aspect, the first composition includes an anionic surfactant and the second composition includes a mixture of zwitterionic and nonionic surfactants.

Numerous cationic surfactants can be used in the compositions described herein. In one aspect, the cationic surfactant can be a quaternary ammonium salt.

Numerous zwitterionic surfactantscan be used in the compositions described herein. In one aspect, the zwitterionic surfactant can be lecithin; in another aspect, the zwitterionic surfactant can be a hydroxysultaine, a betaine, a sulfobetaine, or a mixture thereof. Among betaines, surfactants may be selected from the group comprising high alkyl betaines such as cetyl dimethyl carboxymethyl betaine, cocamidopropyl betaine, cocobetaine, coco dimethyl carboxymethyl betaine, lauryl amidopropyl betaine, lauryl bis-(2-hydroxyethyl) carboxymethyl betaine, lauryl bis-(2-hydroxypropyl)alpha-carboxyethyl betaine, lauryl dimethyl alphacarboxyethyl betaine, lauryl dimethyl carboxymethyl betaine, oleyl betaine, oleyl dimethyl gamma-carboxypropyl betaine, stearyl bis-(2-hydroxypropyl) carboxymethyl betaine, and mixtures thereof. Among sulfobetaines, surfactants may be selected from the group comprising coco dimethyl sulfopropyl betaine, lauryl bis-(2-hydroxyethyl) sulfopropyl betaine, lauryl dimethyl sulfoethyl betaine, stearyl dimethyl sulfopropyl betaine, and mixtures thereof. Amidobetaines and amidosulfobetaines are also contemplated.

Numerous nonionic surfactants can be used in the compositions described herein. Nonionic surfactants useful in the compositions described herein include alkoxylated fatty acid esters, alkyl glucosides, alkyl polyglucosides, amine oxides, cocoamine oxide, glyceryl monohydroxystearate, glyceryl stearate, hydroxy stearic acid, lauramine oxide, laureth-2, polyhydroxy fatty acid amides, polyoxyalkylene stearates, propylene glycol stearate, sorbitan monostearate, sucrose cocoate, sucrose esters, sucrose laurate, steareth-2, and mixtures thereof. Preferred nonionic surfactants include those based on polyethoxylated sorbitan and oleic acid such as, for example, polysorbate 80, which is available under a variety of trade names.

Numerous anionic surfactants can be used herein. In one aspect, the anionic surfactant can be selected from the group comprising alcohol phosphates and phosphonates, alkyl alkoxy carboxylates, alkyl aryl sulfates, alkyl aryl sulfonates, alkyl carboxylates, alkyl ether carboxylates, alkyl ether sulfates, alkyl ether sulfonates, alkyl phosphates, alkyl polyethoxy carboxylates, alkyl polyglucosides, alkyl polyglucoside sulfates, alkyl polyglucoside sulfonates, alkyl succinamates, alkyl sulfates, alkyl sulfonates, aryl sulfates, aryl sulfonates, fatty taurides, isethionates, N-acyl taurates, nonoxynol phosphates, octoxynol phosphates, sarcosinates, sulfated fatty acid esters, taurates, and mixtures thereof. Preferred anionic surfactants include those based upon alkyl polyglucosides as described in U.S. Pat. No. 6,627,612, which is herein incorporated by reference in its entirety. In one aspect, the anionic surfactant is sodium laurylglucosides hydroxypropylsulfonate, available under the trade name Suga®Nate 160 from Colonial Chemical (South Pittsburgh, Tenn.).

In addition to the surfactants with emulsifying properties already discussed, additional emulsifiers can be used herein. In one aspect, the emulsifier can be a polyoxyalkylated fatty acid. In a further aspect, the emulsifier can be polyoxyethylene glyceryl monococoate, polyoxyethylene hydrogenated castor oil, or a mixture thereof. Various other emulsifiers known in the art can be used herein.

In another aspect, one or both compositions of the compositions described herein can include thickeners or viscosity-increasing compounds. Numerous thickenerscan be used. In one aspect, the thickener can be selected from the group comprising cetyl alcohol, glycol stearate, guar gum, gum arabic, hydroxyethylcellulose, polyoxyalkylene distearate, polyoxyalkylene methyl glucose trioleate, polyvinylpyrrolidone, and xanthan gum. A preferred thickener for aqueous compositions is xanthan gum. A particularly preferred form of xanthan gum is Pre-Hydrated® TICAXAN® Rapid-3 Powder available from TIC GUMS (White Marsh, Md.). In one aspect, a thickener for non-aqueous compositions useful herein is polyvinylpyrrolidone (PVP). In another aspect, PVP is povidone K90, which is available from a number of commercial sources.

In a further aspect, one or both compositions can include additional humectants including, but not limited to, aloe vera, caprylyl glycol, colloidal oatmeal, ethylhexylglycerin, hexylene glycol, hydrolyzed collagen protein, hydrolyzed jojoba protein, hydrolyzed wheat protein, mannitol, polyethylene glycols, propylene glycol polymers, sorbitol, sucrose cocoate, urea, xylitol, and mixtures thereof.

In a further aspect, one or both compositions can include an emollient or moisturizer. In one aspect, the emollient compound may also have humectant properties and be selected from the humectants already listed. In another aspect, the emollient can be a straight, branched or cyclic hydroxy compounds such as alcohols containing 1 to 30 carbon atoms; straight, branched, or cyclic carboxylic acids containing 1 to 31 carbon atoms; acid esters containing C₁ to C₃₀ carboxylic acids esterified with C₁ to C₃₀ alcohols; alcohol ethers containing 1 to 30 carbon atoms; alkanes of the formula H—(CH₂)n-H, wherein n is 5 to 30; siloxanes; fats or oils including natural fats and oils such as almond, avocado, beef tallow, castor, coconut, cacao fat, jojoba, lard, mink oils, olive, persic, rice bran, sesame, soybean, hardened oils obtained by hydrogenating the aforementioned oils, and synthetic mono-, di-, and triglycerides such as 2-ethylhexanoic acid glyceride, myristic acid glyceride, and ricinolyl monomaleate triglyceride; waxes such as beeswax, carnauba, lanolin, spermaceti, and derivatives thereof; hydrophobic plant extracts; hydrocarbons such as ceresin, liquid paraffins, microcrystalline wax, petrolatum, pristan, and squalene; higher fatty acids such as behenic, isostearic, lanolic, lauric, linoleic, linolenic, myristic, oleic, palmitic, stearic, and poly unsaturated fatty acids; higher alcohols such as behenyl, cetyl, cholesterol, 2-hexyldecanol alcohol, lauryl, oleyl, and stearyl; esters such as alkyl citrate, alkyl lactate, alkyl tartrate, benzyl laurate, butyl stearate, cetyl lactate, cetyl octanoate, cholesterol isostearate, decyl oleate, glycerol monostearate, glycerol distearate, glycerol tristearate, isopropyl adipate, isopropyl myristate, isopropyl palmitate, myristyl lactate, and myristyl myristate; vitamins such as vitamin A and E, and vitamin alkyl esters, including vitamin C alkyl esters; and mixtures thereof. In a further aspect, the emollient may comprise aliphatic hydrocarbons such as mineral spirits, arachidyl propionate, Carbowax® 300, ethanol, 2-ethylhexyl adipate, 2-ethylhexyl oxystearate, isopropyl myristate, lanolin and lanolin derivatives such as acetylated lanolin and isopropyl lanolate, mineral oil, petroleum jelly, polyoxypropylene cetyl alcohol, polyoxypropylene lanolin alcohol, propionic acid, propylene glycol, stearic acid, stearyl alcohol, and siloxanes such as cyclic polydimethylsiloxane, hexamethyldisiloxane, linear polydimethylsiloxane, poly dimethyl/trimethylsiloxane, polypheylmethylsiloxane, and mixtures thereof. Other phenyl, ethyl and vinyl substituted polysilanes should also be considered to be within the scope of this invention.

In a further aspect, the first and/or second compositions can include essential oils, plant extracts, or other natural products. These may be prepared or collected by any technique known in the art. In one aspect, the plant extracts are purified to concentrate one or more active compounds. In another aspect, the plant extracts are used in a crude state. In a further aspect, the crude or purified plant extracts may exhibit antimicrobial activity. Various plant extracts are contemplated; these may be derived from plants selected from the group comprising the genera Apium, Asperula, Calamintha, Camellia, Carum, Coriandrum, Eucalyptus, Eugenia, Humulus, Hyptis, Lamium, Lycopus, Marrubium, Melaleuca, Mentha, Micromeria, Monarda, Mosla, Myrtus, Nepeta, Ocimum, Origanum, Physostegia, Rosa, Rosmarinus, Salvia, Saturea, Scutellaria, Stachys, Syzygium, Teucrium, Thymus, Trichostema, related, plants and genetic crosses thereof.

Various purified active compounds can be present in the first and/or second compositions. Examples of such compounds include, but are not limited to, acetaldehyde diethyl acetal, anethole, anisaldehyde, berberine, borneol, 2-butanone, camphene, camphor, carvacrol, carvone, carvyl acetate, cedrol, cineol, cinnamaldehyde, cinnamic acid, cinnamic alcohol, cis-4-heptenal, cis-3-hexenol, cis-j asmone, citronellol, cymene, damascenone, damascones, 1-ethoxyethyl acetate, ethyl formate, eucalyptol, eugenol, fenchol, ferulic acid, gamma-nonalactone, gamma-octalactone, geraniol, germacrene D, hexanal, hexanol, hinokitiol, 2-hydroxypropiophenone, isoamyl isovalerate, isobomeol, isobutyraldehyde, isoeugenol, isomenthone, isopulegol, isosafrole, isovaleraldehyde, limonene, linalool, menthofuran, menthol, menthone, menthyl acetate, menthyl methyl ether, menthyl propionate, methyl dihydrojasmonate, methyl salicylate, methyl salicylic acid, mint lactone, myrtenyl acetate, nerol, 3-octanol, 3-octyl acetate, p-cymol, 2-phenylethyl alcohol, 2-phenylethyl isobutyrate, 2-phenylethyl isovalerate, pinene, pinocarvone, piperitone, rhodinol, rose oxide, sabinene hydrate, safrole, terpineol, thymol, 1,3E,5Z-undecatriene, vertenone, viridiflorol, semisynthetic derivatives thereof, and mixtures thereof. A particularly preferred purified active compound is eugenol. In one aspect, one or more of the active compounds listed above can be included in the second composition described herein.

In another aspect, a fragrance or other additive known in the art may be incorporated into either of the compositions described herein. In one aspect, the fragrance may be based on an herb or spice scent, a floral scent, a fruit or vegetable scent, or a mixture thereof. In one aspect, the fragrance is a cinnamon fragrance.

In one aspect, the first composition is composed of water, Suga®Nate 160, copper (II) gluconate, fragrance, and Pre-Hydrated® TICAXAN® Rapid-3 Powder. In a further aspect, the second composition is composed of propylene glycol, glycerin, polysorbate 80, cocamidopropyl betaine, povidone K90, eugenol, and 8-hydroxyquinoline salicylate.

In general, the first and second compositions are kept separate from each other until the time of use. Upon admixing the first and second compositions, a chelation complex between the metal ion present in the first composition and the ligand in the second composition will form. Here, the ligand possesses at least one group capable of chelating with the metal ion. For example, the lone pair electrons present on the nitrogen atom of 8-hydroxyquinoline can chelate with a metal ion (e.g., copper ion).

In one aspect, the first and compositions are dispensed simultaneously on a substrate of interest. In another aspect, the first and second compositions are dispensed sequentially on a substrate of interest. After the first and second compositions are dispensed, they are admixed on the surface or substrate to which they have been applied for a sufficient time for the ligand to chelate the metal ion. Upon sufficient admixture, a color change can occur, indicating formation of the colloidal chelate. In one aspect, this color change may be visible to the eye without the aid of specialized equipment. In another aspect, this color change can optionally include fluorescence. Not wishing to be bound by theory, the metal ion and ligand can react to form various fluorescent compounds which may be visible under ultraviolet light.

In one aspect, the compositions described herein may be used to remove microorganisms from a skin surface of an animal. In a further aspect, that animal may be a mammal. In another aspect, the mammal can be a human. In a further aspect, the skin surface can comprise a hand, wrist, forearm, elbow, face, scalp, foot, ankle, leg, back, abdomen, or any other body surface covered by skin. In an alternative aspect, the compositions disclosed herein may be applied to the hair or the nails.

In another aspect, the compositions described herein may be used to disinfect an inanimate surface or other substrate. In a further aspect, the surface or substrate may be located in a home, a hospital or medical office, a veterinary clinic, an industrial setting, a hotel or restaurant, a ship or boat, a retail store, or an office. In another aspect, the surface or substrate may be a plumbing fixture, a piece of furniture, a counter, a floor, a door or doorknob, a hand rail, an exam table, or any other surface where infectious microorganisms are expected to be present. In a further aspect, the plumbing fixture may be a sink, a toilet, a tub, or a shower stall.

In another aspect, a method of use for the compositions disclosed herein is provided. In a further aspect, this method comprises dispensing the first and second composition onto the surface of a body part and rubbing the surface of the body part for a sufficient time to admix the two compositions. In a further aspect, the body surface may be rubbed for about 2 minutes, about 90 seconds, about 1 minute, about 45 seconds, about 30 seconds, about 20 seconds, about 15 seconds, about 10 seconds, or about 5 seconds. In another aspect, an incidental color change can occur at some point after the admixing of the two compositions, indicating formation of the colloidal chelate. In an additional aspect, after the body surface is rubbed, it may be rinsed with water to remove the compositions described herein, and dried as necessary.

In another aspect, an additional method of use for the compositions disclosed herein is provided. In a further aspect, this method involves dispensing the first and second composition onto the inanimate surface or substrate to be disinfected and scrubbing the surface or substrate for a sufficient time to admix the two compositions. In one aspect, the surface or substrate may be scrubbed for about 2 minutes, about 90 seconds, about 1 minute, about 45 seconds, about 30 seconds, about 20 seconds, about 15 seconds, about 10 seconds, or about 5 seconds. In another aspect, an incidental color change can occur at some point after the admixing of the two compositions, indicating formation of the colloidal chelate. In one aspect, after the surface or substrate is scrubbed, it may be rinsed with water to remove the compositions described herein, and dried as necessary.

In another aspect, the microorganisms to be killed and/or removed from skin surfaces can include bacteria, fungi, archaea, and protozoa. In a further aspect, the compositions described herein canbe effective at removing viruses from skin surfaces, hair or nails, and/or inanimate surfaces and substrates.

In a further aspect, the molecularly-sized nascent state of the chelation complexes formed by the compositions described herein canpenetrate the skin and provide residual antimicrobial and/or antiviral activity. In another aspect, formation of the colloidal chelate indicates that hand washing is complete and that the composition may be washed off with water. Not wishing to be bound by theory, upon admixing the first and second compositions described herein, small colloidal particles form initially that can penetrate the skin. Shortly thereafter, the particles aggregate into larger particles, which are readily washed off the skin with water.

In one aspect, the organism to be killed and/or removed from the skin is a Gram positive bacterium. In a further aspect, the Gram positive bacterium can be from the genera Clostridium, Enterococcus, Listeria, Staphylococcus, and Streptococcus. In another aspect, the organism to be killed and/or removed from the skin is a Gram negative bacterium. In a further aspect, the Gram negative bacterium can be from the genera Bordetella, Borrelia, Brucella, Campylobacter, Escherichia, Francisella, Haemophilus, Helicobacter, Klebsiella, Legionella, Leptospira, Neisseria, Proteus, Pseudomonas, Rickettsia, Salmonella, Shigella, Treponema, Vibrio, and Yersinia. In a further aspect, the organism to be killed and/or removed from the skin is not typically subjected to Gram staining and can be from the genera Chlamydia, Chlamydophila, Mycobacterium, and Mycoplasma.

In another aspect, the organism to be killed and/or removed from the skin is a fungus. In a further aspect, the fungus can be from the genera Aspergillus, Candida, Cryptococcus, Histoplasma, and Pneumocystis.

In an additional aspect, the organism to be killed and/or removed from the skin is a protozoan. In a further aspect, the protozoan can be from the genera Entamoeba, Giardia, Plasmodium, and Toxoplasma.

In another aspect, the compositions described herein can be effective at removing viruses from the skin. In a further aspect, the virus can be from the following families: Adenoviridae, Arenaviridae, Astroviridae, Caliciviridae, Coronaviridae, Filoviridae, Hepadnaviridae, Herpesviridae, Orthomyxoviridae, Papillomaviridae, Paramyxoviridae, Parvoviridae, Picornaviridae, Polyomaviridae, Poxviridae, Reoviridae, Retroviridae, and Rhabdoviridae.

The first and second compositions possess long shelf lives and retain their antimicrobial efficacies after storage prior to admixing. In one aspect, the period of storage can be from about 1 day to about 60 days. In a further aspect, the period of storage may be from about 1 day to about 30 days. In a still further aspect, the period of storage may be about 2 days, about 7 days, about 14 days, about 21 days, or about 28 days.

### Examples

The following examples are put forth so as to provide those of ordinary skill in the art with a complete disclosure and description of how the compositions and methods described and claimed herein are made and evaluated, and are intended to be purely exemplary and are not intended to limit the scope of what the inventors regard as their invention. Efforts have been made to ensure accuracy with respect to numbers (e.g. amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g. component concentrations, desired solvents, solvent mixtures, temperatures, pressures, and other reaction ranges and conditions that can be used to optimize the produce purity and yield obtained from the described process. Only reasonable and routine experimentation will be required to optimize such process conditions.

### Antimicrobial Soap Formulations

The components used to prepare one soap formulation are provided in Table 1. Compositions A and B are stored separately prior to use and mixed at the time of use.

TABLE 1
Antimicrobial Soap Formulation

| | | Parts by Weight | Weight % in |
|---|---|---|---|
| Composition | Component | in Composition | Composition |
| First | Deionized water | 70.00 | 64.49 |
| | Suga ®Nate 160 | 20.00 | 27.64 |
| | Copper (II) gluconate | 4.54 | 4.18 |
| | Fragrance | 3.00 | 2.76 |
| | Rapid-3 prehydrated | 1.00 | 0.92 |
| | xanthan gum | | |
| Second | Propylene glycol | 50.00 | 45.11 |
| | Glycerin | 30.00 | 27.06 |
| | Polysorbate 80 | 10.00 | 9.02 |
| | Cocamidopropyl betaine | 10.00 | 9.02 |
| | Povidone K90 | 5.00 | 4.51 |
| | Eugenol | 3.00 | 2.71 |
| | 8-hydroxyquinoline | 2.85 | 2.57 |
| | salicylate | | |

### Evaluation of Antimicrobial Soap Formulation

The antimicrobial effectiveness of the first and second compositions was assessed over time according to United States Pharmacopeia (USP) method 35-2012. The following steps were performed as part of the testing procedure:
1. Test samples of the first composition and the second composition were inoculated with five different microorganisms (Escherichia coli, Pseudomonas aeruginosa, Staphylococcus aureus, Candida albicans, Aspergillus niger). For bacteria, the inoculum level was 1×10⁶ colony forming units (CFU) per gram and for fungi, the inoculum level was 1×10⁵ colony forming units (CFU) per gram.
2. The inoculated test samples were stored at 20-25° C. for 28 days.
3. The population of each challenge microorganism was determined by the plate count method at Day 2, Day 7, Day 14, Day 21, and Day 28.
4. Plate counts were performed at a 1:10 initial dilution using modified Letheen Broth (LB) as diluent with Tryptic Soy (for bacteria) and Sabouraud Dextrose (for fungi) agar as plating media.

Preservative testing was performed for both the first and the second compositions. According to the test described above, any microbial growth over the allotted time for any of the tested microorganisms means the composition being tested is not effective as a preservative/antimicrobial agent. Results for the first composition are presented in Tables 2, 3, and 4.

TABLE 2
First Composition Preservative Testing (CFU/g)

| | Inoculum/ | Day | Day | Day | Day | Day |
|---|---|---|---|---|---|---|
| Organism | g | 2 | 7 | 14 | 21 | 28 |
| Staphylococcus aureus | 1.70 × 10⁶ | <10 | <10 | <10 | <10 | <10 |
| (bacterium, ATCC | | | | | | |
| #6538) | | | | | | |
| Pseudomonas aeruginosa | 1.70 × 10⁶ | <10 | <10 | <10 | <10 | <10 |
| (bacterium, ATCC | | | | | | |
| #9027) | | | | | | |
| Escherichia coli | 1.70 × 10⁶ | <10 | <10 | <10 | <10 | <10 |
| (bacterium, ATCC | | | | | | |
| #8739) | | | | | | |
| Candida albicans | 1.70 × 10⁵ | <10 | <10 | <10 | <10 | <10 |
| (yeast, ATCC | | | | | | |
| #10231) | | | | | | |
| Aspergillus niger | 1.70 × 10⁵ | <10 | <10 | <10 | <10 | <10 |
| (mold, ATCC | | | | | | |
| #16404) | | | | | | |

TABLE 3
First Composition Preservative Testing (CFU/g)

| Organism | Day 14 | Day 28 |
|---|---|---|
| Staphylococcus aureus | 4.04 | 4.04 |
| Pseudomonas aeruginosa | 4.04 | 4.04 |
| Escherichia coli | 5.09 | 5.09 |
| Candida albicans | 5.07 | 5.07 |
| Aspergillus niger | 5.23 | 5.23 |

TABLE 4
Preservative Testing Validation for First Composition

| | | Dilu- | Microbial | Dil- | Percent |
|---|---|---|---|---|---|
| Organism | Inoculum | tion | Recovery | uent | Recovery |
| Staphylococcus | 39 cfu/plate | 1:10 | 29 cfu/plate | LB | 74% |
| aureus | | | | | |
| Pseudomonas | 50 cfu/plate | 1:10 | 50 cfu/plate | LB | 100% |
| aeruginosa | | | | | |
| Escherichia coli | 32 cfu/plate | 1:10 | 27 cfu/plate | LB | 84% |
| Candida albicans | 50 cfu/plate | 1:10 | 42 cfu/plate | LB | 84% |
| Aspergillus niger | 65 cfu/plate | 1:10 | 52 cfu/plate | LB | 80% |

Based on the results of the preservative testing, the first composition was found to be effective in exerting its antimicrobial effectiveness, and effective in maintaining sterility over the time period tested. Identical tests were performed on the second composition. Results are presented in Tables 5, 6, and 7.

TABLE 5
Second Composition Preservative Testing (CFU/g)

| | Inoculum/ | Day | Day | Day | Day | Day |
|---|---|---|---|---|---|---|
| Organism | g | 2 | 7 | 14 | 21 | 28 |
| Staphylococcus aureus | 1.70 × 10⁶ | <10 | <10 | <10 | <10 | <10 |
| (bacterium, ATCC | | | | | | |
| #6538) | | | | | | |
| Pseudomonas aeruginosa | 1.70 × 10⁶ | <10 | <10 | <10 | <10 | <10 |
| (bacterium, ATCC | | | | | | |
| #9027) | | | | | | |
| Escherichia coli | 1.70 × 10⁶ | <10 | <10 | <10 | <10 | <10 |
| (bacterium, ATCC | | | | | | |
| #8739) | | | | | | |
| Candida albicans | 1.70 × 10⁵ | <10 | <10 | <10 | <10 | <10 |
| (yeast, ATCC | | | | | | |
| #10231) | | | | | | |
| Aspergillus niger | 1.70 × 10⁵ | <10 | <10 | <10 | <10 | <10 |
| (mold, ATCC | | | | | | |
| #16404) | | | | | | |

TABLE 6
Second Composition Preservative Testing (CFU/g)

| Organism | Day 14 | Day 28 |
|---|---|---|
| Staphylococcus aureus | 4.04 | 4.04 |
| Pseudomonas aeruginosa | 4.04 | 4.04 |
| Escherichia coli | 5.09 | 5.09 |
| Candida albicans | 5.07 | 5.07 |
| Aspergillus niger | 5.23 | 5.23 |

TABLE 7
Preservative Testing Validation for First Composition

| | | Dilu- | Microbial | Dil- | Percent |
|---|---|---|---|---|---|
| Organism | Inoculum | tion | Recovery | uent | Recovery |
| Staphylococcus | 39 cfu/plate | 1:10 | 29 cfu/plate | LB | 74% |
| aureus | | | | | |
| Pseudomonas | 50 cfu/plate | 1:10 | 50 cfu/plate | LB | 100% |
| aeruginosa | | | | | |
| Escherichia coli | 32 cfu/plate | 1:10 | 27 cfu/plate | LB | 84% |
| Candida albicans | 50 cfu/plate | 1:10 | 42 cfu/plate | LB | 84% |
| Aspergillus niger | 65 cfu/plate | 1:10 | 52 cfu/plate | LB | 80% |

Based on the results of the preservative testing, the second composition was found to be effective in exerting its antimicrobial effectiveness, and effective in maintaining sterility over the time period tested. Neither the first composition nor the second composition promotes the growth of microorganisms; both compositions disclosed herein are shelf stable.

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

What is claimed:

1. A kit comprising:
a. a first composition comprising a metal salt; and
b. a second composition comprising a ligand, wherein the ligand comprises (i) a residue of an antibacterial agent and (ii) a residue of an antifungal agent, wherein the antibacterial agent and/or antifungal agent comprises at least one aryl group.

2. The kit of claim 1, wherein the metal salt comprises a copper salt, and zinc salt, or manganese salt, or any combination thereof.

3. The kit of claim 1, wherein the first composition comprises the metal salt and water.

4. The kit of claim 1, wherein the metal salt is from 0.1 to 10 parts by weight of the first composition.

5. The kit of claim 1, wherein the antibacterial agent comprises a benzoic acid.

6. The kit of claim 1, wherein the antibacterial agent is salicylic acid.

7. The kit of claim 1, wherein the antifungal agent comprises a quinoline compound.

8. The kit of claim 1, wherein the antifungal agent is 8-hydroxyquinoline.

9. The kit of claim 1, wherein the second composition comprises the ligand and one or more organic solvents.

10. The kit of claim 1, wherein the ligand is from 1 to 10 parts by weight of the second composition.

11. The kit of claim 1, wherein the ligand is produced in situ by reacting the antibacterial agent with antifungal agent.

12. A method for disinfecting a surface of a substrate, the method comprising:
a. applying the first composition and second composition of claim 1 to the surface of the substrate; and
b. admixing the first composition and second composition.

13. A method for disinfecting the skin of a subject, the method comprising:
a. applying the first composition and second composition of claim 1 to the skin of the subject; and
b. admixing the first composition and second composition.
